(19) United States
(12) Patent Application Publication
(10) Pub. No.: US 2022/0098979 A1
(43) Pub. Date: Mar. 31, 2022

(54) 3D RESERVOIR MODELLING BASED ON DYNAMIC PRODUCTION LOGGING TECHNOLOGIES

(71) Applicant: GeoSplit LLC, Moscow (RU)

(72) Inventors: Andrey GURYANOV, Vladimirskaya oblast (RU); Ruslan GAZIZOV, resp. Bashkortostan (RU); Pavel BUZIN, Moskva (RU); Evgeny MALYAVKO, Moskovskaya obl (RU)

(21) Appl. No.: 17/490,403

(22) Filed: Sep. 30, 2021

Related U.S. Application Data
(60) Provisional application No. 63/085,814, filed on Sep. 30, 2020.

(51) Int. Cl. E21B 49/00; E21B 47/11

(52) U.S. Cl. CPC E21B 49/008; E21B 43/26; E21B 2200/20; E21B 47/11

(57) ABSTRACT

Reservoir modelling is achieved via the tracing of inflow profiles of horizontal wells in an oilfield. The tracing is implemented via quantum dot markers released into the wells at specific times. The markers are released out of containers or other release-type tools at different intervals of interest. Each set of markers consists of a specific type of quantum dot that produces light of a specific wavelength when illuminated by ultraviolet (UV) light. The markers contain a hydrophilic material that is attracted to water or an oleophilic material that is attracted to oil. When the markers are flushed out with fluid from the well, the number of markers with a specific identifier associated with a specific interval is detected in the fluid, via the perceived wavelength(s), which, in turn, determine how much water versus oil is produced by each specific interval assessed. Recommendations are provided based on the assessments.

### CROSS-REFERENCE TO RELATED APPLICATIONS

This patent application relates to U.S. Provisional Patent Ser. No. 63/085,814 filed Sep. 30, 2020, all of which is incorporated herein by reference in its entirety.

### FIELD OF THE INVENTION

This invention relates to the field of two- and three-dimensional reservoir modeling.

### BACKGROUND OF THE INVENTION

Horizontal wells, as compared to vertical or directional wells, that develop the same reservoir, have greater production rates, averaging 3-8 times higher production. This allows for reducing operating costs and optimizing the grid of producing wells, and although the number of horizontal wells is rapidly increasing, the production of hydrocarbons is not always as high as the expected, designed volume. For this reason, acquiring the best data on the performance of well production intervals is an important task for operating companies to fully optimize, thereby maximizing efficiency. Moreover, the quality of well completion and reservoir management decisions for production wells largely depends on the production logging data such as mechanical flowmeter and fluid capacitance surveys. Until recently, there has been little alternative to wireline downhole tools for evaluating the placement of fracturing proppant or acids, production rate, and zonal water breakthrough. In today's practice, well intervention in horizontal wells requires implication of coiled tubing or tractor services to deploy logging tools downhole. The success of well intervention depends on several factors and among of these are as follows: well accessibility, completion IDs and length of the horizontal and lateral, etc. There are also key aspects to be considered such as the significant cost of well intervention and the availability of wireline and coiled tubing equipment.

One new alternative method of production logging in horizontal wells is through the utilization of quantum dot markers. The methods and systems of the present invention allow for reducing both cost and the complexities relating to the acquisition of downhole data. Unlike conventional methods, the technology described herein can provide production logs on demand for up to five years after well stimulation via the implementation of quantum dot markers as described.

### SUMMARY OF THE INVENTION

Embodiments of the present disclosure are directed to 2D and 3D reservoir modelling. A 2D/3D reservoir model can be used to estimate a status of an oilfield based on continuously tracing inflow profiles of horizontal oil wells in the oilfield. The tracing can be implemented by placing markers with specific identifiers (i.e., specific sets of quantum dots chemically placed into polymeric microspheres) within interest intervals of oil wells within an oilfield. The markers can be placed inside of containers (i.e., cassettes) or other release-type tools that are then fixed to the lower completion string at different intervals, internally or externally of given pipeline portions. Each container or releasing tool may contain markers comprising a specific set of quantum dots that produces light of a specific wavelength when illuminated by ultraviolet (UV) light. The markers can contain a hydrophilic material that is attracted to water or an oleophilic material that is attracted to oil.

When the markers are flushed out with fluid from the well, the number of markers with a specific identifier associated with a specific interval is detected in the fluid, which determines how much water and oil is produced by the specific interval of the oil well.

The amount of water and oil produced at specific intervals can be used to determine the inflow profiles of the oil wells. The inflow profiles include the volumes of oil produced by the intervals of oil wells, the distributions of the volumes between the intervals, the volumes of water produced by the intervals, and the distribution of volumes of water between the intervals. The inflow profiles are provided to geological models to map residual reserves in the oilfield and/or predict future productivity of the oil wells. Other inputs of the geological models can include permeability and viscosity of the fluid produced by the oil wells, the locations and the geometrical configurations of the oil wells, the intervals of the oil wells in the oilfield, and the geological structure of the formation of the oilfield. The inflow profiles may also be used to provide various recommendations concerning oil recovery improvement. The recommendations may include suggestions for: changes in pressure and rate of oil wells, optimization of pumping pressure and rates of injection wells that maintain reservoir energy, filling the highly conductive channels in the reservoir well with a polymer, and converting the oil well from a production mode to an injection mode.

### BRIEF DESCRIPTION OF THE DRAWINGS

FIGS. 1A-1B show a pipeline with cassettes, placed in a horizontal oil well, according to the present invention. FIG. 1A shows internal cassettes for use in oil wells with cemented completion. FIG. 1B shows external cassettes for use in oil wells having non-cemented completion.

FIG. 2 shows an exemplary inflow profile of a horizontal oil well according to the present invention.

FIG. 3 shows an exemplary classification of inflow profiles according to the present invention.

FIG. 4 shows an exemplary surroundings model according to the present invention.

FIG. 5 shows a flowchart for a method of modelling an oilfield according to the present invention.

FIG. 6 shows an exemplary computer system utilizable by the present invention.

FIG. 7 shows an exemplary 3D mapping of a reservoir according to the present invention.

### DETAILED DESCRIPTION OF THE PREFERRED EMBODIMENT

Embodiments of the present disclosure are directed to 3D reservoir modelling.

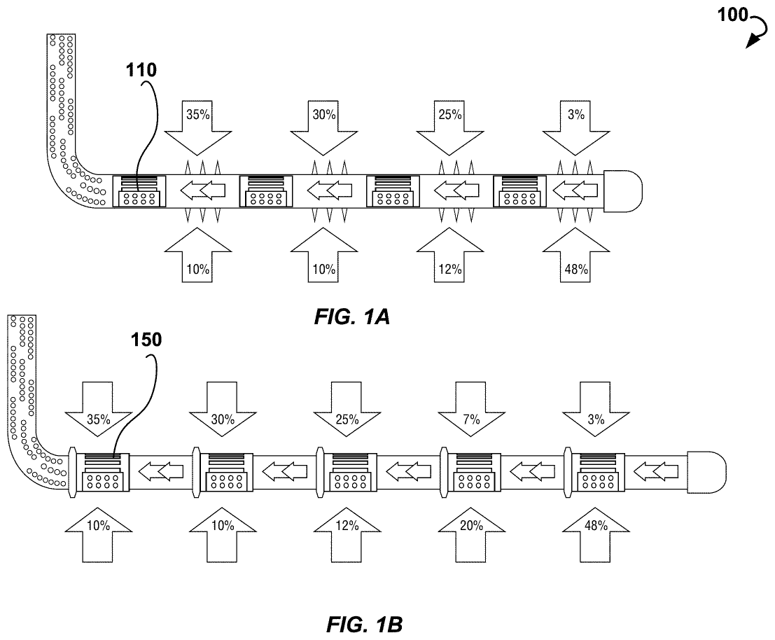

FIGS. 1A and 1B illustrate a pipeline and cassettes with markers placed in a horizontal oil well 100. FIG. 1A shows internal cassettes 110 that can be used in oil wells with cemented completion. The internal cassettes 110 can be inserted into the pipeline. In an example embodiment, a cassette is associated with an interval of a pipeline. In another example embodiment, more than one cassette can be placed at each interval.

FIG. 1B illustrates external cassettes 150 that can be used with those oil wells having non-cemented completion. The external cassettes 150 can be attached to an outer surface of a pipeline in order to enclose the pipeline. In an example embodiment, at least two cassettes can be placed at each interval of a pipeline. The interval length can be in the range of 50-100 meters. The length of a cassette used within an interval can be in the range of 1.5-8 meters. The selection of the length of the cassettes can be based on one or more factors. A cassette can also be filled with a porous material (e.g., a polymer material, sponge-like material, and so forth). The cassette can also be made of materials suitable for acid stimulated wells.

A plurality of markers (i.e., quantum dots, marker-reporters) can be placed into the porous material of the cassette. A marker can comprise microspheres filled with quantum dots. The microspheres can be made of a hydrophilic material or oleophilic material depending on whether the specific microsphere targets water or oil. In particular, the microsphere can be made of an oil- or water-attractable material (e.g., a polymer). Markers that include hydrophilic microspheres are flushed out with water and markers that include oleophilic microspheres are flushed out with oil. The microspheres' quantum dot properties are then identified to determine values desired (including but not limited to a water-to-oil ratio). The microspheres can be about 0.2-1 microns in size depending on the purpose of the microsphere (e.g., for use inside a pipeline or for use between the pipelines).

Each microsphere can be filled with a set of quantum dots. The quantum dots can be associated with a pre-determined code. The set of quantum dots used in the microsphere can be specific to each interval, i.e., they provide a specific signature for that interval from which they were released. Therefore, when different quantum dots are used with different intervals, each set of quantum dots can function as an identifier for specific microspheres because different amounts quantum dots result in different spectra when illuminated by ultraviolet (UV) illumination.

The cassette can have a cylindrical shape surface with openings of predetermined size and shape through which the microspheres can be flushed out from the cassette with fluid. The cassette can include fastening elements configured to secure the cassette to another cassette to form a pipe. The material of the cassette and type of the fastening may depend on a plurality of parameters, such as, for example, whether the pipe or cassettes are assembled at a wellsite or factory site, whether the cassettes are used in non-cemented well completion or cemented well completion, and so forth.

Each cassette may include both hydrophilic microspheres targeting water and oleophilic microspheres targeting oil. When the pipeline is inserted into an oil well, a cassette with markers in the form of microspheres filled with specific sets of quantum dots can be placed within corresponding intervals of the oil well. When a fluid (water or oil) passes through the cassette, the hydrophilic microspheres are flushed out from the cassette with water and oleophilic microspheres are flushed out with oil. Specifically, when the fluid flows through the cassette, the mechanical energy of the fluid flow exceeds the adhesion of the microsphere to a polymer material inside the cassette, and the microsphere disengages from the cassette and flows.

Fluid is then collected from an interval of the oil wells via an array of probes positioned in a predetermined fashion and at predetermined intervals within each oil well. The collected fluid can include two portions, specifically, a water portion and an oil portion disposed above the water portion. Because oil is lighter than the water, water and oil are separated in the probe, therefore, the hydrophilic and oleophilic markers are separated in the probe as well. Specifically, hydrophilic markers (i.e., markers in the form of hydrophilic microspheres) stay in water and oleophilic markers (i.e., markers in the form of oleophilic microspheres) stay in oil in the probe. Thus, the water and oil can be analyzed separately.

Samples of the fluid from each probe are analyzed using a combination of analytical software and hardware. A small diameter liquid jet is formed in this complex. In this flow, the markers are lined up in a row, the passing jet is irradiated with a laser and the marker of each code is identified individually by the signal of light scattering—direct and lateral. Thus, the registered events are counted in each region of the spectral space. Determinations via analysis may include identifying quantity of hydrophilic markers in water and identifying quantity of oleophilic markers in the oil. Such identification can be performed by using UV illumination and based on the color light of the quantum dots. Based on the determined quantity of hydrophilic markers and determined quantity of the oleophilic markers, a quantity of oil and water collected from the interval is determined.

The flow cytometry specifically comprises the following:

When processing the results of cytometry, the interpreter averages the results for all samples and/or divides the samples into series. The series can be divided by, e.g., days of selection, phase composition, modes, or special studies, depending on the task at hand. Series “by day” can be allocated in accordance with a given program. This series type might be formed in the case of strong scatter of sampling dates within a sample; Series “by phase” are formed if the phase composition of the fluid in the sample is very different; Series “by modes” are formed if the sampling was carried out in different technological modes; When carrying out special studies (e.g., sampling during production logging, sampling in different modes), series are allocated in accordance with the given program requirements. Additionally, when analyzing samples, statistical processing of cytometric results is used, the particular method of statistical processing being selected depending on the sample sample (e.g., Levey-Jennings maps, Q-test, Romanovsky test, Dixon test, Irwin test, Charlier test).

The cassettes can be configured to stay in the pipeline and release markers for up to 5 years or longer without the need for removal, reinstallation, or maintenance, as statistical data is continued to be collected and the program is updated. The cassettes can be configured to release markers continuously as long as the reservoir fluid flows through the cassettes.

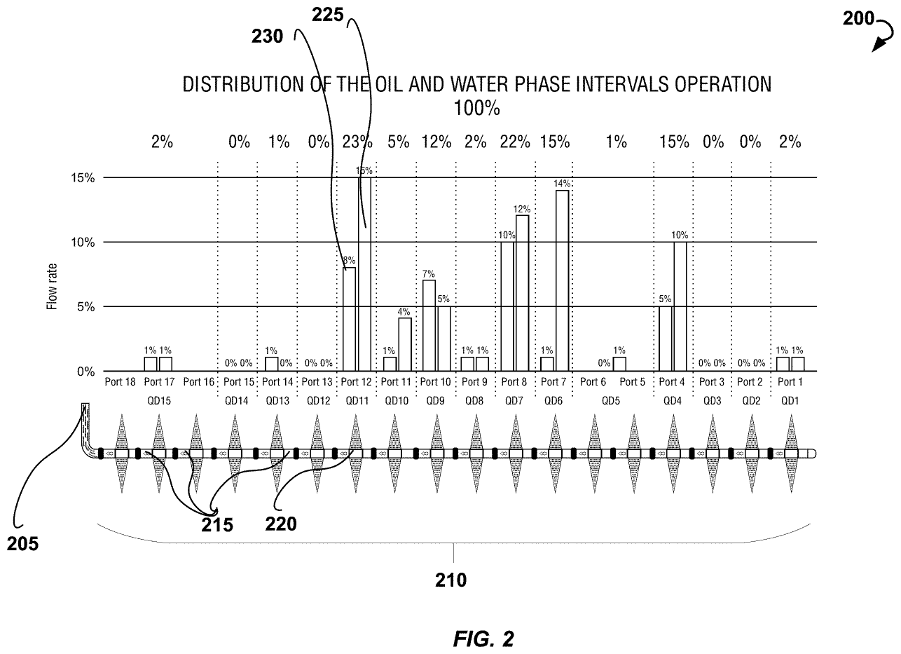

FIG. 2 illustrates an example inflow profile 200 of a horizontal oil well. The inflow profile 200 has a distribution of oil flow rate and water flow rate at each interval of the horizontal oil well. A pipeline has a vertical section 205 and a horizontal section 210. The horizontal section 210 has a plurality of intervals 215. A cassette 220 can be attached to each of the intervals 215. The inflow profile 200 shows a quantity 225 (flow rate) of water and a quantity 230 (flow rate) of oil collected from each of the intervals 215. The inflow profile 200 may visualize the intervals 215 working inefficiently. As can be seen in FIG. 2, based on cytometric analysis, the total amount of oil is produced mostly by four intervals shown as intervals 4, 7, 10, and 12.

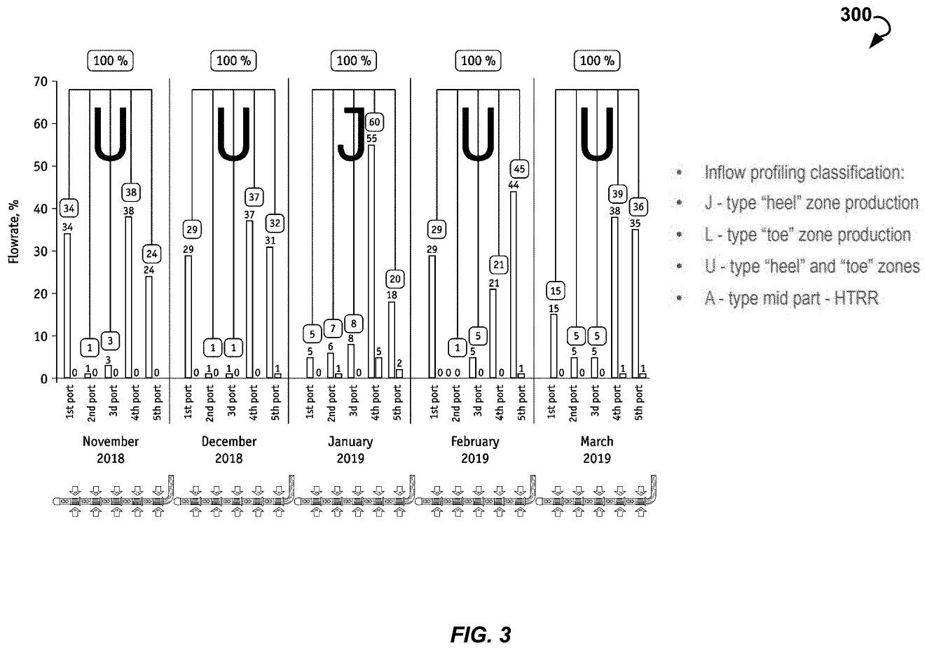

FIG. 3 illustrates an example classification of inflow profiles 300. The inflow profiles 300 show oil flow profiles and water flow profiles of intervals of a horizontal oil well. The horizontal section of the well may have a heel zone located closer to the vertical section of the well, a toe zone located at a distal end with respect to the vertical section, and a middle zone located between the heel zone and the toe zone. The inflow profiles 300 may show the profiles of production of oil by the same intervals. The profiles may include a J-type profile when the oil is produced predominantly by the intervals in the heel zone, an L-type profile when the oil is produced predominantly by the intervals in the toe zone, a U-type profile when the oil is produced predominantly by the intervals in the heel zone and the toe zone, and an A-type profile when the oil is produced predominantly by the intervals in the middle zone of the interval.

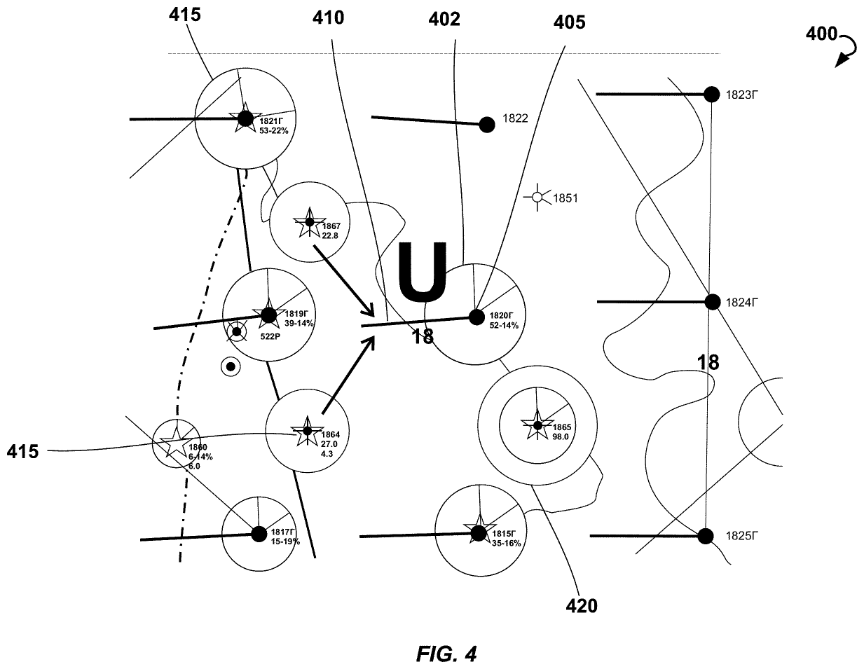

FIG. 4 illustrates an example of a map of modelled surroundings of an oil well area. The map is created on the basis of drilling data, geophysical studies of the area, inclinometry of the area, and any project documents (e.g., the point of entry into the formation, the direction of the trunk, the distance between wells, etc.). A surroundings model (i.e., map) 400 may include locations of the nearest injection wells. Specifically, a surroundings model 400 may include modelled locations of a well 402, a vertical section 405, and a horizontal section 410 of the well. The well 402 may include a U-type profile of oil production, as shown in this Figure. Upon analysis of the surroundings model 400, it can be further determined that several injection wells 415 maintain formation pressure closer to the toe portion of the pipeline and there is an injection well 420 that maintains formation pressure closer to the heel portion of the pipeline.

Based on the analysis of the surroundings model 400, recommendations concerning the operation of the well can be provided. The recommendations can include, for example, filling some of the injection wells with polymers (to redistribute oil or water production channels), drilling an additional injection well nearby the horizontal well, turning one of the production horizontal wells into the injection well, and so forth.

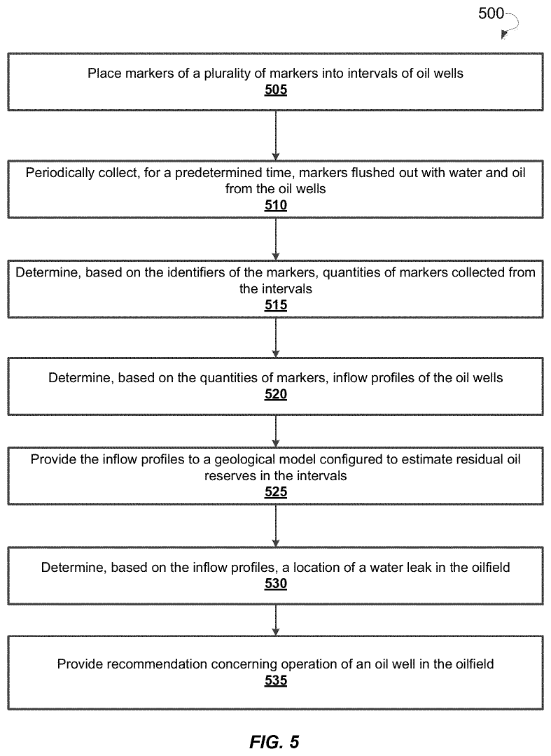

FIG. 5 is a flow chart of a method 500 for modeling of an oilfield. The method 500 may commence with the placing of a plurality of markers into intervals of oil wells at step 505, via microspheres as described above. Each quantum dot within a microsphere includes identifiers which are unique to a specific interval within the oil wells, as determined prior to installation. This uniqueness of each microsphere via quantum dots is formed by the generation of a unique code that is associated each quantum dot. Each cassette comprises a polymer compound and a plurality of one identifiable type of quantum dot, thereby relating markers to their interval of origin/installation. At step 510, markers can be flushed out with water and oil from the oil wells and collected periodically for a predetermined time, wherein collection is implemented via probes of the fluid from the wells. In an example embodiment, an artificial intelligence (AI) model can use machine-learning techniques and can be trained using neural networks to identify certain types of quantum dots, thereby automatically determining the intervals associated with the identifiers.

Therefore, after horizontal wells (production wells or injection wells) are drilled in oil reservoirs, the production wells may be tested using markers placed in cassettes associated with specific intervals. Specific markers can be used for specific intervals of the wells. The markers are flushed out with fluid, collected in a probe from the well, and analyzed as described herein.

The method 500 can further include determining quantities of markers collected from the intervals based on the identifiers of the markers at step 515. The process of identifying the quantities of markers within a probe of fluid can be represented by three general stages:

1) Concentrated sample preparation from the probe of formation fluid (comprised of crude oil in pure form, formation saline water, and various unique markers). This process makes it possible to extract markers in a concentrated form from the formation fluid probe through sequential processes of sorption and desorption of the unique markers within the probe using a special material. The final stage of this concentrated sample preparation process is the desorption of markers into an aqueous medium to obtain a concentrated sample of various unique markers. Each probe received by the laboratory is subject to this first step, such that concentrated samples containing various unique markers are created for step 2.

2) Cytometric analysis. The concentrated samples obtained as a result of the concentrated sample preparation are analyzed using a flow cytometer for cytometric analysis of the concentrated samples. The result of this analysis is a determination of a value representing a number of distinct markers within the probe of fluid, divided into parts by means within the flow cytometer, wherein each marker has its unique code. Analysis of the fluorescence identified during the cytometric analysis determines which unique markers are present and in what amounts. This interval marker data is used to identify/categorize the inflow profiles of each researched interval, e.g., as required for multistage hydraulic fracturing.

3) Further processing of the interval marker data. The results of the cytometric analysis are presented in the form of diagrams, the diagrams representing the flow profile for all researched intervals.

The results of analysis include percentages of oil and water in the intervals. At step 520, inflow profiles of the oil wells can be determined based on the quantities of markers. An inflow profile of the oil wells can include volumes of oil produced by intervals of the oil well and volumes of water produced by the intervals of the oil well.

The method 500 can continue with generating a geological model. The inflow profiles can be provided to the geological model at step 525. The geological model can be configured to estimate residual oil reserves in the intervals. The geological model can depend on one or more of the following: locations of the intervals of the oil wells in the oilfield, arrangement of the intervals of the oil wells with respect to each other, a geological structure of the formation of the oilfield, lithological, physical, and facial characteristics of reservoir rock, and so forth. The geological model can be used to determine, e.g., current and potential states of the wells, influence of horizontals wells and injection wells on each other, predict future productivity of the horizontal wells, estimate an amount of oil in a reservoir, determine current and potential states of development of a reservoir, evaluate water flooding efficiency, create a map of fluid flows consumed by different wells, and so forth.

The analysis performed by using the geological model may include performing classification of the wells, determining influence of the wells on each other, determining structure of the oil reservoir in terms of location of water field and oil fields and location of water leakage, estimating the environment of the wells (i.e., the structure of rock surrounding the wells), estimating the future production of the wells, predicting the production of the wells by simulation of shutting down some of the wells and converting some of the wells from production to injection, providing recommendations concerning optimal operation of the wells, for example, converting a production well into an injection well, shutting down the well, measures for increasing the percentage of oil or water production, reasons as to why the interval provided the determined percentage of oil and water, prediction with regard to productiveness of the oil well in a shelf, interrelation of different oil wells, and so forth. The recommendations may be provided based on computer modelling, simulation, and so forth.

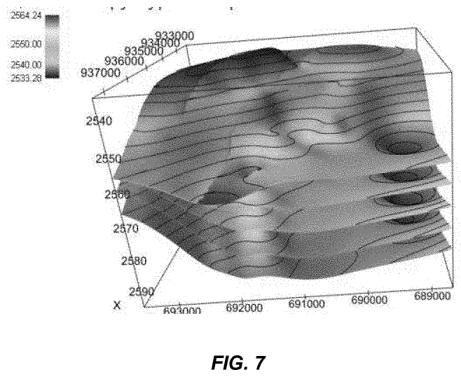

In some embodiments, a plurality of parameters may be considered by the geological model. The parameters can include properties of fluids, permeability of fluids, geology, structure of the shelf, and so forth. Based on the analysis and 2-D model, and using a 3-D mapping tool, a three-dimensional map of the surroundings of oil wells can be generated for further optimization. The 3-D map can visualize the structure of the shelf and the amounts of fluids in the shelf. See, e.g., FIG. 7. The map may further visualize the percentage of oil and water produced by each interval, influence of wells on each other, fluid flows consumed by different wells, and so forth.

At step 530, a location of a water leak in the oilfield can be determined based on the inflow profiles. At step 535, at least one recommendation concerning operation of an oil well in the oilfield can be provided. The recommendation can be based on inflow profiles, location of the water leak, and residual oil reserves in the intervals. The recommendation can include one of the following: shutting down the oil well, filling the oil well with a polymer, converting the oil well from a production mode into an injection mode, and so forth.

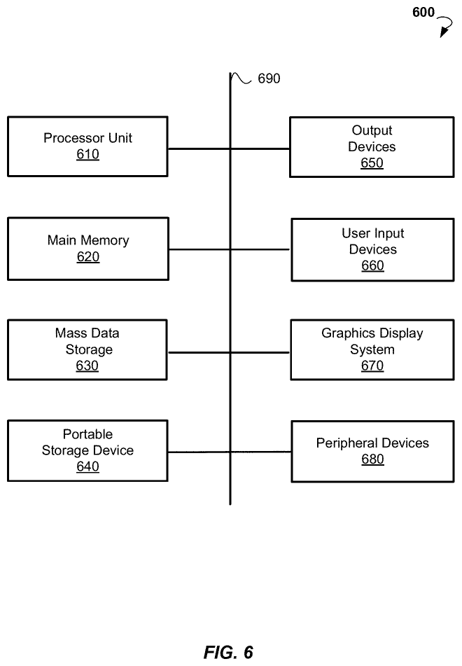

FIG. 6 illustrates an exemplary computer system 600 that may be used to implement some embodiments of the present invention. The computer system 600 of FIG. 6 may be implemented in the contexts of the likes of computing systems, networks, servers, or combinations thereof. The computer system 600 of FIG. 6 includes one or more processor units 610 and main memory 620. Main memory 620 stores, in part, instructions and data for execution by processor units 610. Main memory 620 stores the executable code when in operation, in this example. The computer system 600 of FIG. 6 further includes a mass data storage 630, portable storage device 640, output devices 650, user input devices 660, a graphics display system 670, and peripheral devices 680.

The components shown in FIG. 6 are depicted as being connected via a single bus 690. The components may be connected through one or more data transport means. Processor unit 610 and main memory 620 is connected via a local microprocessor bus, and the mass data storage 630, peripheral device(s) 680, portable storage device 640, and graphics display system 670 are connected via one or more input/output (I/O) buses.

Mass data storage 630, which can be implemented with a magnetic disk drive, solid state drive, or an optical disk drive, is a non-volatile storage device for storing data and instructions for use by processor unit 610. Mass data storage 630 stores the system software for implementing embodiments of the present disclosure for purposes of loading that software into main memory 620.

Portable storage device 640 operates in conjunction with a portable non-volatile storage medium, such as a flash drive, floppy disk, compact disk, digital video disc, or Universal Serial Bus (USB) storage device, to input and output data and code to and from the computer system 600 of FIG. 6. The system software for implementing embodiments of the present disclosure is stored on such a portable medium and input to the computer system 600 via the portable storage device 640.

User input devices 660 can provide a portion of a user interface. User input devices 660 may include one or more microphones, an alphanumeric keypad, such as a keyboard, for inputting alphanumeric and other information, or a pointing device, such as a mouse, a trackball, stylus, or cursor direction keys. User input devices 660 can also include a touchscreen. Additionally, the computer system 600 as shown in FIG. 6 includes output devices 650. Suitable output devices 650 include speakers, printers, network interfaces, and monitors.

Graphics display system 670 include a liquid crystal display (LCD) or other suitable display device. Graphics display system 670 is configurable to receive textual and graphical information and processes the information for output to the display device.

Peripheral devices 680 may include any type of computer support device to add additional functionality to the computer system.

The components provided in the computer system 600 of FIG. 6 are those typically found in computer systems that may be suitable for use with embodiments of the present disclosure and are intended to represent a broad category of such computer components that are well known in the art. Thus, the computer system 600 of FIG. 6 can be a personal computer (PC), handheld computer system, telephone, mobile computer system, workstation, tablet, phablet, mobile phone, server, minicomputer, mainframe computer, wearable, or any other computer system. The computer may also include different bus configurations, networked platforms, multi-processor platforms, and the like. Various operating systems may be used including UNIX, LINUX, WINDOWS, MAC OS, PALM OS, QNX, TIZEN, ANDROID, IOS, CHROME, and other suitable operating systems.

The processing for various embodiments may be implemented in software that is cloud-based. In some embodiments, the computing system 200 is implemented as a cloud-based computing environment, such as a virtual machine operating within a computing cloud. In other embodiments, the computing system 200 may itself include a cloud-based computing environment, where the functionalities of the computing system 200 are executed in a distributed fashion. Thus, the computing system 200, when configured as a computing cloud, may include pluralities of computing devices in various forms, as will be described in greater detail below.

In general, a cloud-based computing environment is a resource that typically combines the computational power of a large grouping of processors (such as within web servers) and/or that combines the storage capacity of a large grouping of computer memories or storage devices. Systems that provide cloud-based resources may be utilized exclusively by their owners or such systems may be accessible to outside users who deploy applications within the computing infrastructure to obtain the benefit of large computational or storage resources.

The cloud may be formed, for example, by a network of web servers that comprise a plurality of computing devices, such as the computing device 200, with each server (or at least a plurality thereof) providing processor and/or storage resources. These servers may manage workloads provided by multiple users (e.g., cloud resource customers or other users). Typically, each user places workload demands upon the cloud that vary in real-time, sometimes dramatically. The nature and extent of these variations typically depends on the type of business associated with the user.

The analysis and recommendation methods of the present invention comprise the following:

1) Clarification and updating of the geological structure of the reservoir areas, which includes conducting lithological and facies analysis and geological and reservoir modelling of the formation;

2) Analysis of the current state of reserves, recovery status, development, and production (e.g., dynamics of development indicators, reservoir pressure analysis, analysis of well-flooding factors);

3) Evaluation of the flooding system effectiveness and the calculation of a degree of flow communication between wells using the Spearman's rank correlation method;

4) Geological and field analysis of the dynamic marker-based production logging data and analysis of the factors responsible for the changing dynamics of flow profiles over time; also, analysis of interference and the presence of a single flow communication system; and

5) Development of a set of recommendations for managing the development process and improving the efficiency of the reservoir pressure maintenance system.

Geological and field analysis of well performance and reserves recovery in the drainage area based on marker-based production surveillance further enables the ability to quantify the contribution of each frac port to a horizontal well's performance, thereby assisting in developing a decision-making algorithm for optimizing a given field development system.

The marker-based technology involves obtaining a stream of data on the flow profile and composition for each studied interval of a horizontal wellbore by adding marker-reporters directly during the injection of proppant, throughout the course of multi-stage hydraulic fracturing, or by integrating the marker-reporters into lower completions, wherein the marker-reporters are placed in special marker containers/cassettes during well construction, thereafter being released from the containers/cassettes as reservoir fluid flows through the containers/cassettes. Each frac stage, through the distinctive marker-reporters added during that stage, corresponds to a unique marker-reporter code representing the stage. When contacting the reservoir fluid in the course of well operation, the marker-reporters are released from the polymer matrix and washed out by the fluid flow to the surface. During well operation, fluid samples are taken at the wellhead at a certain frequency. The samples are then analyzed, as described above, to quantify the contribution of each interval to the overall well production for each reservoir fluid phase.

Based on the analysis of the results of dynamic marker-based production surveillance in horizontal wells, an algorithm of consistent decision-making for regulating the reserve development process may be developed and applied to a specific reservoir system. The integrated approach combines express methods for development analysis and the results of marker-based production logging to enable prompt decision-making on the regulation and maintenance of any field development system. The provided set of recommendations for optimizing the field development process, in turn, leads to better well production/performance, as well as an increase in water flooding efficiency (e.g., by blocking high permeability intervals, and by reaching previously undrained reservoir areas).

### Example

The present invention was implemented via a geological and field feasibility study of the Yuzhno-Vyintoiskoye reservoir for field development and management. Marker-reporter-based production logging enabled prompt decision-making to adjust the field development system at current/instantaneous stages.

After implementing conformance control measures for the first field within the reservoir, the water content stabilises at 32% without significant growth. The conformance control measures enabled a reduction of the rate of oil production decline. The total increment in oil production across the first field varied from 11 tons/day to 14 tons/day (in the first and last months, respectively). The effectiveness evaluation of the implemented technology showed that a positive economic effect was obtained at the subject field. Given the uninterrupted well operation, the cumulative additional oil production amounted to 1,430 tons, and the effect continues to date.

For the second field within the reservoir, recommendations were issued as early actions to prevent injected water breakthroughs to production wells, as well as to further substantiate flooding areas to boost production. Based on the generalisation, systematisation, and analysis of the results of the dynamic marker-reporter-based production logging in the horizontal wells, an algorithm for monitoring the field and for consistent decision-making was developed, thereby improving the management of the recovery of hydrocarbon reserves at the particular field.

Thus, the set of recommendations for optimizing the field development process, provided via the present invention, facilitated the achievement of higher production/performance, as well as an increase in water flooding efficiency through the blocking of high permeability intervals and the reaching of previously undrained areas within the reservoir.

The present technology is described above with reference to example embodiments. Therefore, other variations upon the example embodiments are intended to be covered by the present disclosure.

The description of a preferred embodiment of the invention has been presented for purposes of illustration and description. It is not intended to be exhaustive or to limit the invention to the precise forms disclosed. Obviously, many modifications and variations will be apparent to practitioners skilled in this art. It is intended that the scope of the invention be defined by the following claims and their equivalents.

Moreover, the words “example” or “exemplary” are used herein to mean serving as an example, instance, or illustration. Any aspect or design described herein as “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

What is claimed is:

1. A method for analyzing and improving oilfield production, comprising:
introducing a plurality of microspheres into an oil well during a given fracturing event, a portion of said plurality of microspheres comprising hydrophilic microspheres, another portion of said plurality of microspheres comprising oleophilic microspheres, wherein all microspheres carry within them an identifiable plurality of quantum dots,
periodically collecting, at collection points, fluid of the oilfield, said fluid comprising oil, water, and said microspheres,
determining, based on identifiers of said quantum dots within said collected fluid, one or more quantities of microspheres,
determining, based on said one or more quantities of microspheres, one or more inflow profiles of said oilfield,
determining, based on said one or more inflow profiles, a water leak location of said oilfield,
providing, based on said one or more inflow profiles and/or any determined water leak locations, one or more recommendations for improving the oilfield production.

2. The method of claim 1, further comprising:
combining said one or more inflow profiles with a geological model of said oilfield to create a surroundings map, and
providing said one or more recommendations based on said surroundings map.

3. The method of claim 2, wherein said geological model comprises additional data, said additional data including a permeability and a viscosity of said fluid, locations and geometrical configurations of oil wells within said oilfield, intervals of said oil wells within said oilfield, and a geological structure of a formation of the oilfield.

4. The method of claim 1, where said one or more recommendations comprises changes in a pressure and a rate of one or more wells within said oilfield.

5. The method of claim 1, where said one or more recommendations comprises changes to a pumping pressure and rates of injection wells within said oilfield.

6. The method of claim 1, where said one or more recommendations comprises filling highly conductive channels within said oilfield with a polymer.

7. The method of claim 1, where said one or more recommendations comprises converting one or more wells within said oilfield from a production mode to an injection mode, or vice versa.

8. The method of claim 1, wherein said one or more recommendations comprises adding an injection well.

9. The method of claim 1, wherein said one or more inflow profiles comprises data on volumes of oil produced by given intervals of oil wells within said oilfield, distributions of said volumes of oil produced, volumes of water produced, and distributions of said volumes of water produced.

10. The method of claim 1, wherein said introducing said plurality of microspheres comprises installing one or more cassettes, said cassettes comprising a polymer material and said plurality of microspheres.

11. The method of claim 10, wherein said cassettes are external cassettes.

12. The method of claim 10, wherein said cassettes are internal cassettes.

13. The method of claim 1, further comprising:
classifying said one or more inflow profiles as one of J-type, L-type, or U-type.

14. The method of claim 1, wherein said one or more inflow profiles of the oilfield are determined using a flow cytometer.

15. The method of claim 14, further comprising presenting results of an analysis using said flow cytometer in one or more diagrams, the one or more diagrams representing a flow profile for any set of oilfield intervals.
